(19) United States
(12) Patent Application Publication
(10) Pub. No.: US 2008/0166812 A1
(43) Pub. Date: Jul. 10, 2008

(54) Method of using a meter to determine an analyte concentration

(75) Inventor: John P. Creaven, Granger, IN (US)

Correspondence Address: NIXON PEABODY LLP, 161 N. CLARK STREET, 48TH FLOOR, CHICAGO, IL, 60601, US

(73) Assignee: Bayer HealthCare LLC

(21) Appl. No.: 11/906,895

(22) Filed: Oct. 4, 2007

Related U.S. Application Data
(60) Provisional application No. 60/878,953, filed on Jan. 5, 2007.

(51) Int. Cl. G01N 33/00; B01J 19/00

(52) U.S. Cl. 436/2; 422/68.1; 73/866

(57) ABSTRACT

A method of using a meter to determine an analyte concentration of a fluid sample. The method includes providing a meter with stored default calibration information and stored generic calibration information. The method further includes providing a package including at least one test sensor to receive a fluid sample to be analyzed. The method additionally includes placing the package into the meter and determining whether the received package includes an auto-calibration circuit or label. If the test sensor does not include the auto-calibration circuit or label, the method utilizes the stored default calibration information in the meter to determine the analyte concentration of the sample received by the test sensor. If the test sensor includes the auto-calibration circuit or label, the method utilizes the auto-calibration circuit or label and the stored generic calibration information to determine the analyte concentration of the sample received by the test sensor. The method further includes placing the fluid sample on the test sensor and determining the analyte concentration of the sample.

### CROSS-REFERENCE TO RELATED APPLICATIONS

This application claims priority to U.S. Provisional Application Ser. No. 60/878,953 filed on Oct. 5, 2006, which is incorporated by reference in its entirety.

### FIELD OF THE INVENTION

The present invention relates generally to methods and systems for the elimination of a certain percentage of auto-calibration circuits or labels. The auto-calibration circuits or labels are used in automatically calibrating instruments or meters that determine the concentration of an analyte (e.g., glucose) in a fluid.

### BACKGROUND OF THE INVENTION

The quantitative determination of analytes in body fluids is of great importance in the diagnoses and maintenance of certain physiological abnormalities. For example, lactate, cholesterol, and bilirubin should be monitored in certain individuals. In particular, determining glucose in body fluids is important to diabetic individuals who must frequently check their blood glucose levels to regulate the glucose intake in their diets. The results of such tests can be used to determine what, if any, insulin or other medication needs to be administered. In one type of blood-glucose testing system, sensors are used to test a sample of blood.

A test sensor contains biosensing or reagent material that reacts with, for example, blood glucose. The testing end of the sensor is adapted to be placed into the fluid being tested, for example, blood that has accumulated on a person's finger after the finger has been pricked. The fluid is drawn into a capillary channel that extends in the sensor from the testing end to the reagent material by capillary action so that a sufficient amount of fluid to be tested is drawn into the sensor. The fluid then chemically reacts with the reagent material in the sensor resulting in an electrical signal indicative of the glucose level in the fluid being tested. This signal is supplied to the meter via contact areas located near the rear or contact end of the sensor and becomes the measured output.

Diagnostic systems, such as blood-glucose testing systems, typically calculate the actual glucose value based on a measured output and the known reactivity of the reagent-sensing element (test sensor) used to perform the test. The reactivity or lot-calibration information of the test-sensor is provided on a calibration circuit that is associated with the sensor package. This calibration circuit is typically physically inserted by the end user. In other cases, the calibration is automatically done using an auto-calibration circuit via a label on the sensor package. In this case, calibration is transparent to the end user and does not require the end user to insert a calibration circuit into the meter. Manufacturing millions of sensor packages, each having a calibration circuit or label to assist in calibrating the sensor package, can be expensive. Thus, there exists a need to reduce the cost associated with calibrating the sensor packages.

### SUMMARY OF THE INVENTION

According to one aspect of the present invention, a method of using a meter adapted to determine an analyte concentration of a fluid sample is disclosed. The method comprises the act of providing a meter with stored default calibration information and stored generic calibration information. The method further includes the act of providing a package including at least one test sensor adapted to receive a fluid sample to be analyzed. The method additionally comprises the acts of placing the package into the meter and determining whether the received package includes an auto-calibration circuit or label. If the test sensor does not include the auto-calibration circuit or label, the method includes the act of utilizing the stored default calibration information in the meter to assist in determining the analyte concentration of the fluid sample received by the test sensor. If the test sensor includes the auto-calibration circuit or label, the method includes the act of utilizing the auto-calibration circuit or label and the stored generic calibration information to assist in determining the analyte concentration of the fluid sample received by the test sensor. The method further includes the acts of placing the fluid sample on the test sensor and determining the analyte concentration of the fluid sample.

According to another aspect of the present invention, a method of manufacturing a package adapted to contain at least one test sensor is disclosed. The method comprises the act of providing at least one test sensor in the package. The test sensor is adapted to receive a fluid sample to be analyzed. The method also includes the acts of testing selected information associated with the at least one test sensor, assigning specific calibration information to the package based on the testing of selected information, and determining whether the specific calibration information of the at least one test sensor corresponds to predetermined calibration information. If the specific calibration information does not correspond to the predetermined calibration information, the method further includes the act of providing an auto-calibration circuit or label on the package.

According to yet another aspect of the present invention, a system for determining an analyte concentration reading of a fluid sample is disclosed. The system comprises a package adapted to contain at least one test sensor. The at least one test sensor is adapted to receive the fluid sample to be analyzed. The system further comprises a meter having stored default calibration information. The meter is adapted to receive the package and determine whether the package includes the auto-calibration circuit or label. The meter is also adapted to (a) utilize the stored default calibration data to assist in analyzing the fluid sample if the package does not include the auto-calibration circuit or label, and (b) utilize the auto-calibration circuit or label to assist in analyzing the fluid sample if the package includes the auto-calibration circuit or label.

According to yet another aspect of the present invention, a method of preparing a test sensor for use with a meter to determine an analyte concentration is disclosed. The method comprises the act of providing a test sensor adapted to receive a fluid sample to be analyzed. The method further comprises the acts of testing selected information associated with the test sensor, assigning specific calibration information to the test sensor based on the testing of selected information, and determining whether the specific calibration information of the test sensor corresponds to predetermined calibration information. The method also comprises the act of providing an auto-calibration code chip if the specific calibration information does not correspond to the predetermined calibration information.

The above summary of the present invention is not intended to represent each embodiment, or every aspect, of the present invention. Additional features and benefits of the present invention are apparent from the detailed description and figures set forth below.

### BRIEF DESCRIPTION OF THE DRAWINGS

FIG. 1 shows a sensing instrument according to one embodiment.

FIG. 2 shows the interior of the sensing instrument of FIG. 1.

FIG. 3 shows a sensor package according to one embodiment for use with the sensing instrument of FIG. 2.

FIG. 4 shows a sensor package according to another embodiment for use with the sensing instrument of FIG. 2.

FIG. 5A shows a sensing instrument and a sensor cartridge according to an alternative embodiment.

FIG. 5B shows the sensor cartridge of FIG. 5A.

FIG. 6A show a sensing instrument and a sensor cartridge according to another alternative embodiment.

FIG. 6B shows the sensor cartridge of FIG. 6A.

FIG. 7A shows a single test-sensor meter according to one embodiment.

FIG. 7B shows a code chip adapted for use with the single test-sensor meter of FIG. 7A.

While the invention is susceptible to various modifications and alternative forms, specific embodiments are shown by way of example in the drawings and are described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATED EMBODIMENTS

Generally, an instrument or meter uses a test sensor adapted to receive a fluid sample to be analyzed, and a processor adapted to perform a predefined test sequence for measuring a predefined parameter value. A memory is coupled to the processor for storing predefined parameter data values. Calibration information associated with the test sensor may be read by the processor before the fluid sample to be measured is received. Calibration information may be read by the processor after the fluid sample to be measured is received, but not after the analyte concentration has been determined. Calibration information is generally used to compensate for different characteristics of test sensors, which will vary on a batch-to-batch basis. In some systems, the specific calibration information is provided on an auto-calibration circuit or label that is associated with each test sensor batch. The present invention contemplates methods and systems for eliminating a percentage of auto-calibration circuits or labels while maintaining accurate meter readings.

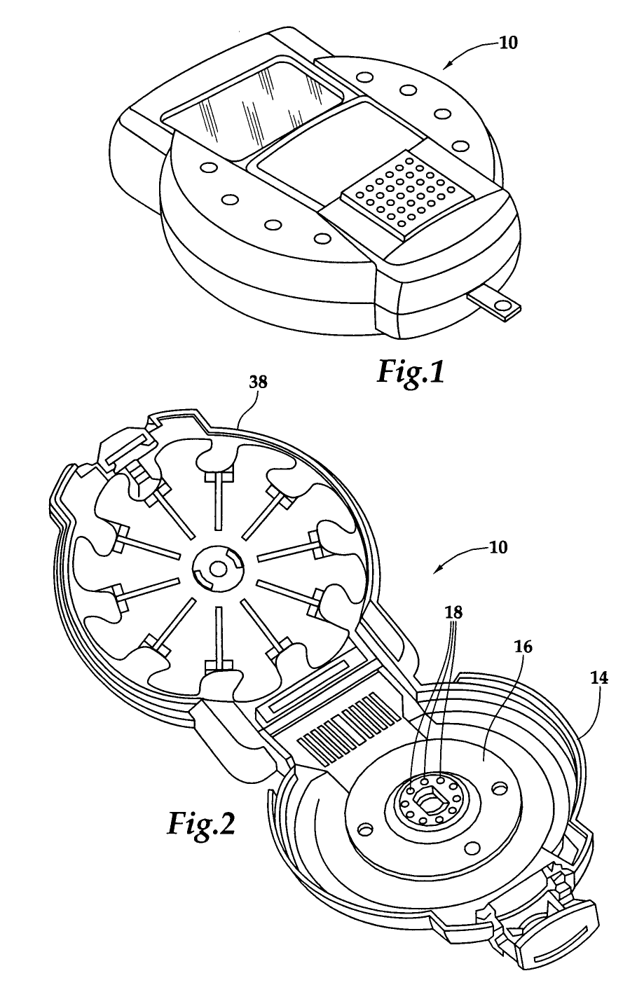

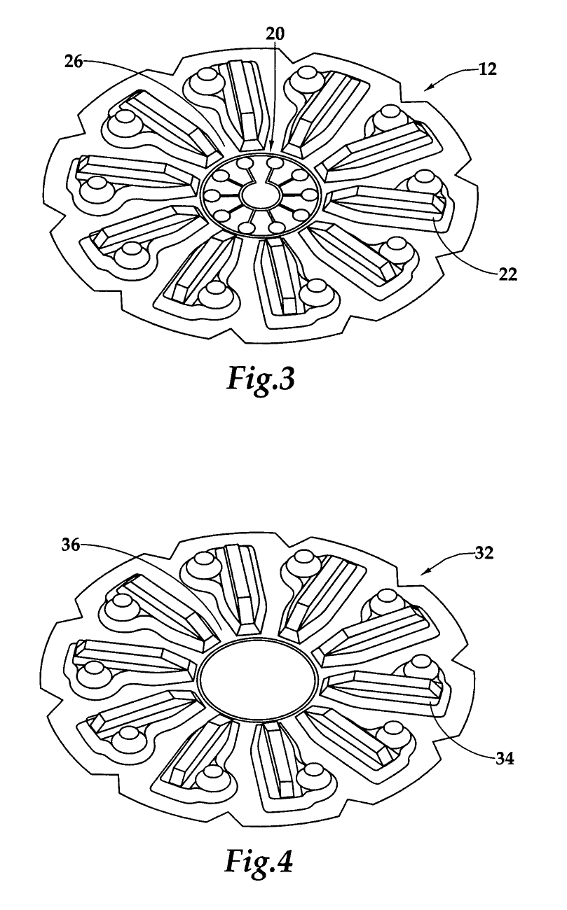

More specifically, referring now to FIGS. 1 and 2, an instrument or meter 10 is illustrated. In FIG. 2, the inside of the instrument 10 is shown in the absence of a sensor package. Examples of sensor packages (sensor packages 12 and 32) are separately illustrated in FIGS. 3 and 4. Referring back to FIG. 2, a base member 14 of the instrument 10 supports an auto-calibration plate 16 and a predetermined number of auto-calibration pins 18. As shown in FIG. 2, for example, the instrument 10 includes ten auto-calibration pins 18. It is contemplated that the number of auto-calibration pins may vary in number and shape from that shown in FIG. 2. The auto-calibration pins 18 are connected for engagement with the sensor package 12 (FIG. 3).

The sensor package 12 of FIG. 3 includes an auto-calibration circuit or label 20, a plurality of test sensor pockets 22 including test sensors (not illustrated), and a sensor-package base 26. The plurality of test sensors 22 is used to determine concentrations of analytes. Analytes that may be measured include glucose, lipid profiles (e.g., cholesterol, triglycerides, LDL and HDL), microalbumin, hemoglobin A_{1C}, fructose, lactate, or bilirubin. It is contemplated that other analyte concentrations may be determined. The analytes may be in, for example, a whole blood sample, a blood serum sample, a blood plasma sample, other body fluids like ISF (interstitial fluid) and urine, and non-body fluids. As used within this application, the term “concentration” refers to an analyte concentration, activity (e.g., enzymes and electrolytes), titers (e.g., antibodies), or any other measure concentration used to measure the desired analyte.

The plurality of test sensors 22 includes an appropriately selected enzyme to react with the desired analyte or analytes to be tested. An enzyme that may be used to react with glucose is glucose oxidase. It is contemplated that other enzymes may be used such as glucose dehydrogenase. An example of a test sensor is disclosed in U.S. Pat. No. 6,531,040 assigned to Bayer Corporation. It is contemplated that other test sensors may be used.

Specific calibration information or codes assigned for use in the clinical value computations to compensate for manufacturing variations between sensor lots are encoded on the auto-calibration circuit or label 20. The auto-calibration circuit or label 20 is used to automate the process of transferring the specific calibration information (e.g., the lot specific reagent calibration information for the plurality of test sensors 22) such that the test sensors 22 may be used with at least one instrument or meter. Selected information associated with the test sensor (which varies on a batch-to-batch basis) is tested to determine the specific calibration information to be assigned to the auto-calibration circuit or label 20.

The auto-calibration pins 18 of the meter 10 electrically couple with the auto-calibration circuit or label 20 when a cover 38 of the instrument 10 is closed. The meter 10 includes generic calibration information, typically in the form of an algorithm or equation (e.g., slope and intercept). The generic calibration information in the meter 10 uses the specific calibration information assigned to the calibration circuit or label 20, resulting in finalized integrated calibration information to be used by the meter with this test sensor.

Referring now to FIG. 4, a sensor package 32 is illustrated without the auto-calibration circuit or label 20. Thus, the sensor package 32 does not include any calibration information readable by the meter 10. Otherwise, the sensor package 32 is similar to the sensor package 12, having a plurality of test sensors 34 and a sensor package base 36. The sensor package 32 may also be used with the meter 10. To produce accurate meter readings when the meter 10 is used with the sensor package 32, the meter 10 utilizes stored default calibration information, as will be described below in more detail. The stored default calibration information is defined by certain predetermined calibration information associated with some test sensors.

The predetermined calibration information is used to determine whether a sensor package should include an auto-calibration circuit or label such as, for example, the auto-calibration circuit or label 20. The predetermined calibration information may be arbitrarily selected or systematically selected. In one method, the calibration information history of sensor packages is reviewed over a selected period of time to determine the calibration information to be used as the predetermined calibration information. This time period is typically a longer period of time such that more calibration information is included in the data. For example, the time period may be 6 months, 1 year or even at least 2 years. After reviewing the history of such calibration information assignment, the most common calibration information assigned to the auto-calibration circuits or labels may be determined and utilized, as will be described below. Determining the most common calibration information assigned to the auto-calibration circuits or labels enables an educated “prediction” of the calibration information to be associated with a given test sensor. However, it is not necessary that the most common calibration information be utilized. The present invention contemplates the use of any calibration information that may be assigned to the auto-calibration circuit or label as the predetermined calibration information.

In one example, the specific calibration information included on the auto-calibration circuits or labels 20 may be a program number having a two digit number (coded in a binary format, i.e., 1 or 0 to be read by the meter) that depends on different characteristics of test sensors, which may vary on a batch-to-batch basis. It is contemplated that other calibration information may be used, such as optical detection in the meter of holes or specific marks on the sensor, a code chip inserted into a meter, or a lookup table in the meter to set program number. Thus, each package 12 (with test sensors 22) including an auto-calibration circuit or label 20 has a specific program number (e.g., 36) coded onto the auto-calibration circuit or label 20. The most common program number coded on auto-calibration circuits or labels 20 can be determined from a review of program number statistics over a selected period of time. In some embodiments, a range of common program numbers (e.g., 35-37) can be determined and utilized. However, if a range is utilized, a range sufficient to produce minimal percentage error is encouraged. It is thus desirable to have a larger number of common program numbers while at the same time having a minimal percentage error (less than 2% or 3% may be acceptable). Thus, the predetermined calibration information may be a single program number (e.g., 36), which would have no error, or a range of program numbers (e.g., 35-37), which would desirably have a minimal percentage error.

The predetermined calibration information is utilized in the following way. When a new batch of test sensors is tested, and specific calibration information associated with the batch is determined, the specific calibration information associated with the batch is compared to the predetermined calibration information. If the specific calibration information of this particular batch corresponds to the predetermined calibration information, no auto-calibration circuit or label is provided on the package. However, if the specific calibration information of this particular batch does not correspond to the predetermined calibration information, an auto-calibration circuit or label, such as auto-calibration circuit or label 20, is provided on the package.

The default calibration information stored in the meter corresponds with the predetermined calibration information so as to produce an accurate meter analyte concentration when an auto-calibration circuit or label is not provided on a test sensor package (e.g., package 32). As such, the meter is adapted to determine whether an auto-calibration circuit or label is provided on a package once the package is placed in the meter. If no auto-calibration circuit or label is present, the meter utilizes the default calibration information (which applies the predetermined calibration information) to determine the concentration of an analyte. If the auto-calibration circuit or label is present, the meter utilizes other generic calibration information and the specific calibration information included in the auto-calibration circuit or label to determine the concentration of an analyte.

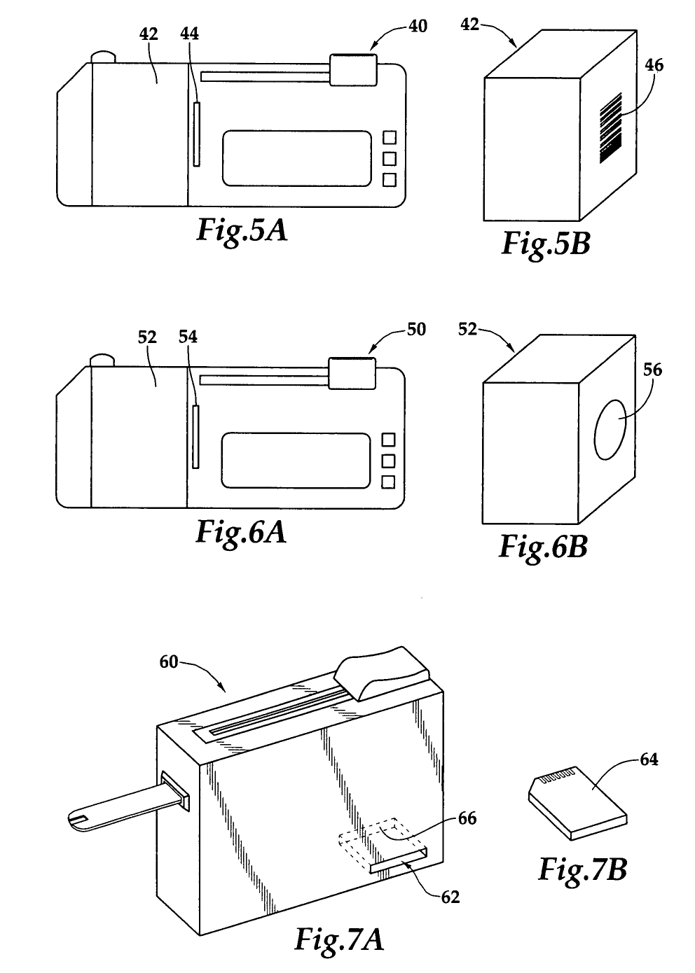

The present invention is not limited to use with the packages 12 and 32 and the meter 10. The present invention may be incorporated with other types of sensor containers and meters. For example, referring now to FIGS. 5A and 5B, an alternative sensor-dispensing instrument 40 and cartridge 42 are illustrated. The sensor-dispensing instrument 40 is adapted to receive the cartridge 42 holding at least one test sensor. The cartridge 42 includes a bar code 46 having calibration information.

In accordance with the present invention as described above, a bar code is not provided on the cartridge if the specific calibration information associated with the at least one test sensor contained in the cartridge falls within the predetermined calibration information range. A bar code is provided if the specific calibration information falls outside the predetermined calibration information range. The sensor-dispensing instrument 40 includes a bar-code reader 44 adapted to read the bar code 46 (if provided) on the cartridge 42 once the cartridge 42 is inserted into the instrument 40. If no bar code is provided, the sensor-dispensing instrument 40 uses stored default calibration information compatible with the predetermined calibration information to achieve an accurate reading of the instrument 40. If a bar code is provided, the bar-code reader 44 reads the bar code 46 to obtain the specific calibration information and uses this bar code calibration information with other generic calibration information in the meter to have finalized calibration information that is used to determine the analyte concentration.

Referring now to FIGS. 6A and 6B, an additional sensor-dispensing instrument 50 and package 52 are illustrated. The sensor-dispensing instrument 50 is adapted to receive the package 52 holding at least one test sensor. The package 52 includes a chip 56 having specific calibration information relating to the test sensor. The instrument 50 is also adapted to receive a package without a chip (not illustrated). The instrument 50 includes stored default calibration data corresponding to predetermined calibration information. If the chip (e.g., chip 56) is provided on the package, such as the package 52, the meter reads the specific calibration information included in the chip to provide the meter reading. If the package does not include a chip, the meter uses the stored default calibration information and the predetermined calibration information to determine and provide the meter reading.

The present invention may also be utilized in single test-sensor embodiments. FIG. 7A illustrates a single test-sensor meter or instrument 60 forming a slot 62 therein adapted to receive a code chip 64 (FIG. 7B). In accordance with the present invention as described above, a code chip (e.g., code chip 64) is not provided with a test sensor if the specific calibration information associated with the test sensor falls within the predetermined calibration information range. A code chip is provided with the test sensor if the specific calibration information falls outside the predetermined calibration information range. The single test-sensor meter 60 includes a reader 66 adapted to read a code chip (if provided) once the code chip is inserted into the slot 62 of the single test sensor meter 60. If no code chip is provided, the single test-sensor meter (e.g., single test sensor meter 60) uses stored default calibration information compatible with the predetermined calibration information to achieve an accurate meter reading. If a code chip is provided, the reader 66 reads the code chip to obtain the specific calibration information and uses the code chip calibration information with other generic calibration information in the meter to have finalized calibration information that is used to determine the analyte concentration.

### Alterative Process A

A method of using a meter adapted to determine an analyte concentration of a fluid sample, the method comprising the acts of:

providing a meter with stored default calibration information and stored generic calibration information;

providing a package including at least one test sensor adapted to receive a fluid sample to be analyzed;

placing the package into the meter;

determining whether the received package includes an auto-calibration circuit or label;

if the test sensor does not include the auto-calibration circuit or label, utilizing the stored default calibration information in the meter to assist in determining the analyte concentration of the fluid sample received by the test sensor;

if the test sensor includes the auto-calibration circuit or label, utilizing the auto-calibration circuit or label and the stored generic calibration information to assist in determining the analyte concentration of the fluid sample received by the test sensor;

placing the fluid sample on the test sensor; and

determining the analyte concentration of the fluid sample.

### Alternative Process B

The method of Alternative Process A, wherein the fluid sample comprises at least one of a whole blood sample, a blood serum sample, a blood plasma sample, interstitial fluid, urine, and other body fluids.

### Alternative Process C

The method of Alternative Process A, wherein the fluid sample comprises a non-body fluid.

### Alternative Process D

The method of Alternative Process A, wherein the analyte is glucose.

### Alternative Process E

The method of Alternative Process A, wherein the auto-calibration circuit or label includes specific calibration information.

### Alternative Process F

The method of Alternative Process E, wherein the specific calibration information is a number.

### Alternative Process G

A method of manufacturing a package adapted to contain at least one test sensor, the method comprising the acts of:

providing at least one test sensor in the package, the test sensor being adapted to receive a fluid sample to be analyzed;

testing selected information associated with the at least one test sensor;

assigning specific calibration information to the package based on the testing of selected information;

determining whether the specific calibration information of the at least one test sensor corresponds to predetermined calibration information; and

providing an auto-calibration circuit or label on the package if the specific calibration information does not correspond to the predetermined calibration information.

### Alternative Process H

The method of Alternative Process G, wherein the auto-calibration circuit or label includes a bar code.

### Alternative Process I

The method of Alternative Process G, wherein the auto-calibration circuit or label includes a chip.

### Alternative Process J

The method of Alternative Process G, wherein the specific calibration information is a number.

### Alternative Embodiment K

A system for determining an analyte concentration reading of a fluid sample, the system comprising:

a package adapted to contain at least one test sensor, the at least one test sensor being adapted to receive the fluid sample to be analyzed; and

a meter having stored default calibration information, the meter being adapted to receive the package and determine whether the package includes the auto-calibration circuit or label, the meter being further adapted to (a) utilize the stored default calibration data to assist in analyzing the fluid sample if the package does not include the auto-calibration circuit or label, and (b) utilize the auto-calibration circuit or label to assist in analyzing the fluid sample if the package includes the auto-calibration circuit or label.

### Alternative Embodiment L

The system of Alternative Embodiment K, wherein the auto-calibration circuit or label includes a bar code.

### Alternative Embodiment M

The system of Alternative Embodiment K, wherein the auto-calibration circuit or label includes a chip.

### Alternative Embodiment N

The system of Alternative Embodiment K, wherein the auto-calibration circuit or label includes specific calibration information.

### Alternative Process O

A method of preparing a test sensor for use with a meter to determine an analyte concentration, the method comprising the acts of:

providing a test sensor adapted to receive a fluid sample to be analyzed;

testing selected information associated with the test sensor;

assigning specific calibration information to the test sensor based on the testing of selected information;

determining whether the specific calibration information of the test sensor corresponds to predetermined calibration information; and

providing an auto-calibration code chip if the specific calibration information does not correspond to the predetermined calibration information.

### Alternative Process P

The method of Alternative Process O, wherein the analyte is glucose.

### Alternative Process Q

The method of Alternative Process O, wherein the specific calibration information is a number.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention.

1. A method of using a meter adapted to determine an analyte concentration of a fluid sample, the method comprising the acts of:
providing a meter with stored default calibration information and stored generic calibration information;
providing a package including at least one test sensor adapted to receive a fluid sample to be analyzed;
placing the package into the meter;
determining whether the received package includes an auto-calibration circuit or label;
if the test sensor does not include the auto-calibration circuit or label, utilizing the stored default calibration information in the meter to assist in determining the analyte concentration of the fluid sample received by the test sensor;
if the test sensor includes the auto-calibration circuit or label, utilizing the auto-calibration circuit or label and the stored generic calibration information to assist in determining the analyte concentration of the fluid sample received by the test sensor; placing the fluid sample on the test sensor; and
determining the analyte concentration of the fluid sample.

2. The method of claim 1, wherein the fluid sample comprises at least one of a whole blood sample, a blood serum sample, a blood plasma sample, interstitial fluid, urine, and other body fluids.

3. The method of claim I, wherein the fluid sample comprises a non-body fluid.

4. The method of claim 1, wherein the analyte is glucose.

5. The method of claim 1, wherein the auto-calibration circuit or label includes specific calibration information.

6. The method of claim 5, wherein the specific calibration information is a number.

7. A method of manufacturing a package adapted to contain at least one test sensor, the method comprising the acts of:
providing at least one test sensor in the package, the test sensor being adapted to receive a fluid sample to be analyzed;
testing selected information associated with the at least one test sensor;
assigning specific calibration information to the package based on the testing of selected information;
determining whether the specific calibration information of the at least one test sensor corresponds to predetermined calibration information; and
providing an auto-calibration circuit or label on the package if the specific calibration information does not correspond to the predetermined calibration information.

8. The method of claim 7, wherein the auto-calibration circuit or label includes a bar code.

9. The method of claim 7, wherein the auto-calibration circuit or label includes a chip.

10. The method of claim 7, wherein the specific calibration information is a number.

11. A system for determining an analyte concentration reading of a fluid sample, the system comprising:
a package adapted to contain at least one test sensor, the at least one test sensor being adapted to receive the fluid sample to be analyzed; and
a meter having stored default calibration information, the meter being adapted to receive the package and determine whether the package includes the auto-calibration circuit or label, the meter being further adapted to (a) utilize the stored default calibration data to assist in analyzing the fluid sample if the package does not include the auto-calibration circuit or label, and (b) utilize the auto-calibration circuit or label to assist in analyzing the fluid sample if the package includes the auto-calibration circuit or label.

12. The system of claim 11, wherein the auto-calibration circuit or label includes a bar code.

13. The system of claim 11, wherein the auto-calibration circuit or label includes a chip.

14. The system of claim 11, wherein the auto-calibration circuit or label includes specific calibration information.

15. A method of preparing a test sensor for use with a meter to determine an analyte concentration, the method comprising the acts of:
providing a test sensor adapted to receive a fluid sample to be analyzed;
testing selected information associated with the test sensor;
assigning specific calibration information to the test sensor based on the testing of selected information;
determining whether the specific calibration information of the test sensor corresponds to predetermined calibration information; and
providing an auto-calibration code chip if the specific calibration information does not correspond to the predetermined calibration information.

16. The method of claim 15, wherein the analyte is glucose.

17. The method of claim 15, wherein the specific calibration information is a number.
